(19) United States
(12) Patent Application Publication
(10) Pub. No.: US 2004/0176730 A1
(43) Pub. Date: Sep. 9, 2004

(54) Disposable, safe butterfly needle sheath

(76) Inventor: Hsien Tsung Wang, Taipei (TW)

Correspondence Address: Hsien Tsung WANG, 4Fl. No. 27, Lane 160,, Hsin Sheng S. Rd., Sec. 1, Taipei, 100, TW

(21) Appl. No.: 10/797,100

(22) Filed: Mar. 11, 2004

(30) Foreign Application Priority Data
Aug. 13, 2002 (TW) 091118217
Aug. 15, 2002 (CN) 02125746.9

(51) Int. Cl. A61M 5/00

(52) U.S. Cl. 604/263

(57) ABSTRACT

A disposable, safe needle sheath assembly comprises a barrel unit including a hollow barrel, a forward extension adapted to insert through a wing unit into a cap, and a T-shaped slot longitudinally formed thereon, a transverse bar of the slot including a locking device in a right side, and a needle unit including a hollow plunger, a needle, two opposite latched members raised above an edge of a rear end, and a circumferential protrusion, the protrusion being projected from the slot after inserting the needle unit in the barrel unit. Removing the cap and sliding the protrusion from the transverse bar to a distal end of the slot will project the needle from the extension for dispensing medicine. To the contrary sliding the protrusion from the distal end of the slot to the locking device will lock the protrusion therein.

### BACKGROUND OF THE INVENTION

1. Field of The Invention

The present invention relates to hypodermic needles and more particularly to a butterfly needle sheath assembly adapted to couple to an intravenous (IV) infusion set. After its use, a needle can be concealed and locked prior to throwing away the needle sheath assembly for safety reasons.

2. Description of Related Art

A conventional butterfly needle sheath assembly for use in cooperation with an IV infusion is shown in FIG. 10. In use, a sheath C is removed prior to piercing a needle N into the body for dispensing fluid through a barrel K (i.e., hypodermic injection). Also, wings B are attached to the body. After the injection, a medical worker should carefully insert the used needle N into the sheath C again.

However, the prior art suffered from several disadvantages. In detail, it is known that an IV infusion may take several hours. It is possible that the needle N may be removed and pierced again in the IV infusion process due to many reasons. Thus, finding the original sheath C for putting on the needle N is required even the removal time of the needle N is short and unfortunately, such may bother a medical worker. Further, the medical worker may be pricked by the needle N if sufficient care is not taken since a diameter or the needle N is very small (e.g., typically less than 1 mm). Also, the needle N may be contaminated by microorganisms or blood after use. This is not safe. Typically, a used needle is either bent or retracted within the interior cavity of a syringe plunger. However, such is a time consuming process. Also, the retracted needle can be used again by pulling the needle from the syringe plunger. This is highly unsafe. Moreover, add a safety device on the existing butterfly needle sheath assembly is very difficult since the needle sheath assembly is small (i.e., no additional space) and is made of plastic material of highly flexible (i.e., structural limitation). Hence, a need for improvement exists.

### SUMMARY OF THE INVENTION

It is an object of the present invention to provide a disposable, safe butterfly needle sheath assembly which, after use, a hypodermic needle can be concealed and a protrusion of a needle unit can be locked in a cavity or opening of a barrel unit prior to throwing away the needle sheath assembly. By utilizing this, a possibility of accidentally pricking a medical worker by the needle after use is substantially eliminated and also it can completely prevent criminals from selling the butterfly needle sheath assemblies to hospitals again by simply cleaning or even disinfecting the used needles.

The above and other objects, features and advantages of the present invention will become apparent from the following detailed description taken with the accompanying drawings.

### BRIEF DESCRIPTION OF THE DRAWINGS

FIG. 1 is a perspective view of a first preferred embodiment of butterfly needle sheath assembly according to the invention;

FIG. 2 is an exploded view of the sheath assembly in FIG. 1;

FIGS. 2A and 2B are detailed views of the areas in right and left circles in FIG. 2 respectively;

FIGS. 3A, 3B and 3C are plan views showing three different locations of protrusion in the short bar of T-shaped slot during and after use of the needle;

FIG. 4 is a side view in part section showing a snapping coupling of the sheath assembly and an IV infusion set in one configuration, where the cap is put on an extension for covering the needle;

FIG. 4A is a view similar to FIG. 4, where the cap is removed and the needle is forwarded;

FIG. 5 is a side view in part section showing a threaded coupling of the sheath assembly and an IV infusion set in another configuration, where the cap is put on the extension for covering the needle;

FIG. 5A is a view similar to FIG. 5, where the cap is removed and the needle is forwarded;

FIG. 6 is a perspective view of a second preferred embodiment of butterfly needle sheath assembly according to the invention;

FIGS. 7A, 7B and 7C are plan views showing three different locations of the protrusion in the short bar of T-shaped slot during and after use of the needle according to the second preferred embodiment;

FIGS. 8 and 9 are sectional views taken along line 8-8 of FIG. 7A and line 9-9 of FIG. 7C respectively; and

FIG. 10 is a top view of a conventional butterfly needle assembly.

### DETAILED DESCRIPTION OF THE PREFERRED EMBODIMENTS

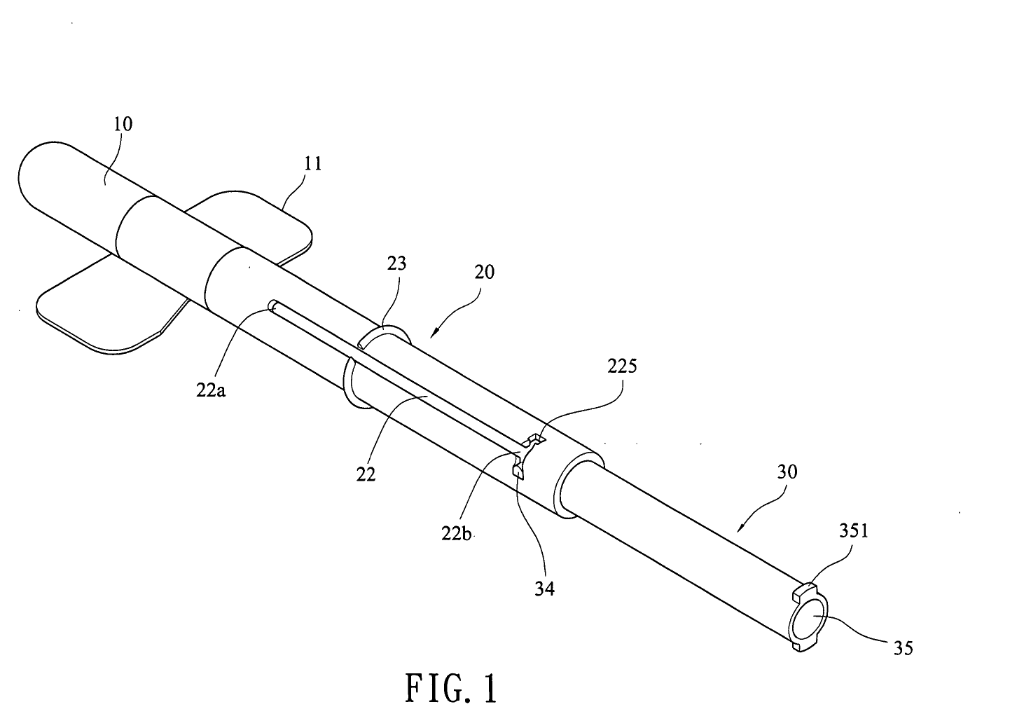

Referring to FIGS. 1 to 5, there is shown a first preferred embodiment of butterfly needle sheath assembly constructed in accordance with the invention. The sheath assembly comprises a cap 10, a wing unit 11, a barrel unit 20, and a needle unit 30. Each component will be described in detail below.

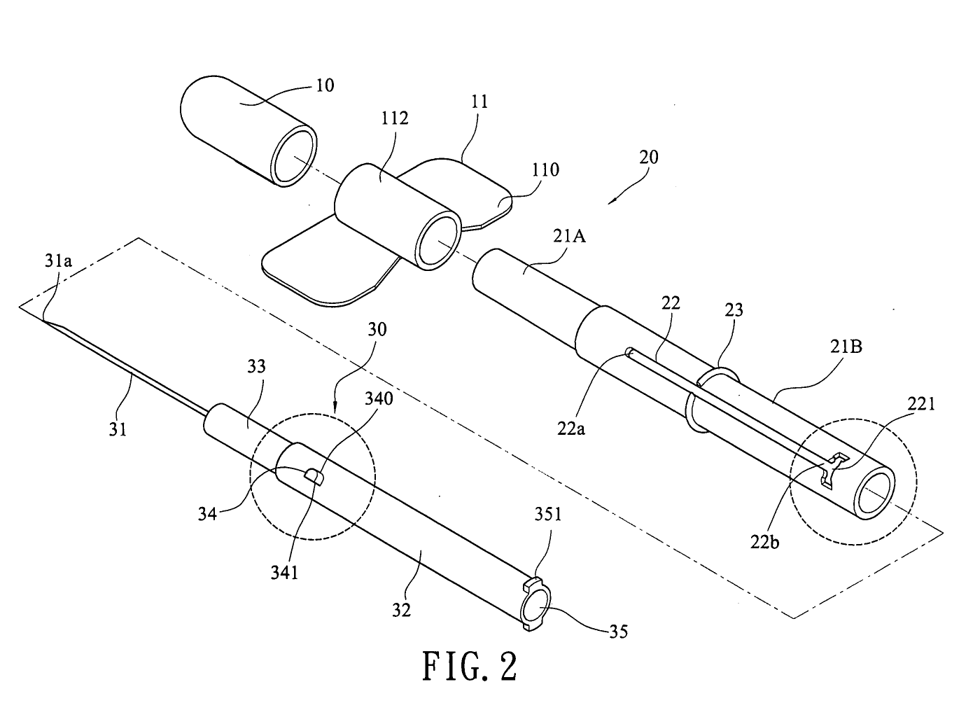

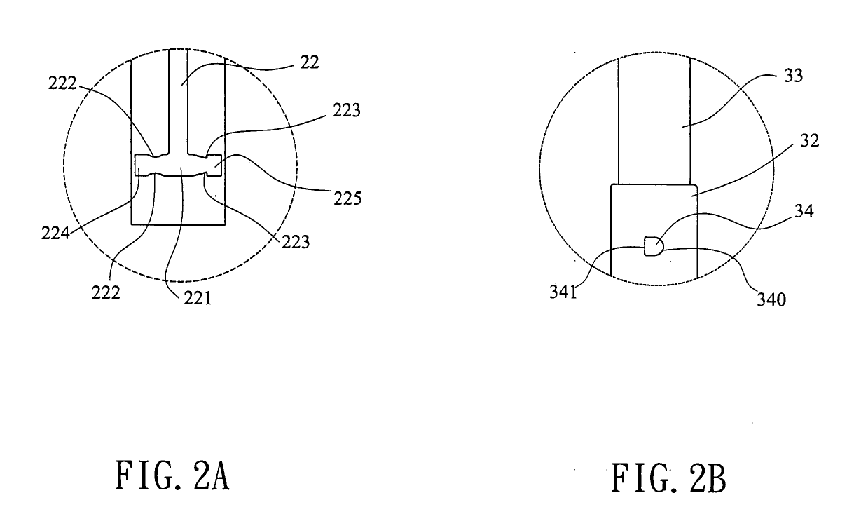

The wing unit 11 comprises a hollow cylinder 112 and two wings 110 at both sides of the cylinder 112. The wings 110 are attached to the body in hypodermic injection as well known in the art. The hollow barrel 20 including a hollow, cylindrical extension 21A at a forward end of the barrel 20, the extension 21A having a smaller outer diameter to be able to snugly insert through the cylinder 112 into the cap 10 for coupling, a body 21B having an outer diameter the same as that of the cylinder 112, and a T-shaped slot 22 longitudinally formed on an outer surface of the body 21B, the slot 22 including a forward stop 22a at the distal end of the long bar and a turning point 22b at the joining point of the long bar and a short bar 221. A slip-resistant ring 23 is formed around an intermediate portion of the body 21B. As shown in FIG. 2A, the short bar 221 comprises a left throat 222, a left end 224, a right shoulder 223, and a cavity 225 at the right end.

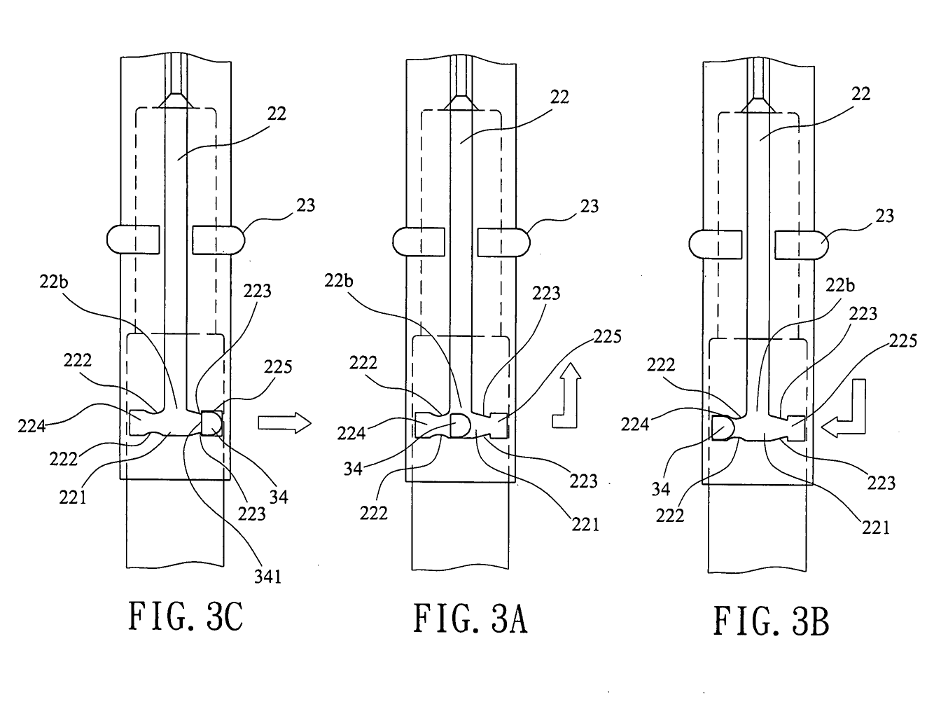

The needle unit 30 comprises a hollow plunger 32, a forward extension 33 having a smaller diameter as compared to that of the plunger 32, a needle 31 protruded from the extension 33, two opposite latched members 351 raised above the edge of a rear end 35, and a protrusion 34 projected from an outer surface, the protrusion 34 including a curved section 340 and a straight section 341. The needle unit 30 is adapted to insert in the barrel unit 20 in which the protrusion 34 is projected from and slidable along the slot 22, and the needle 31 is adapted to retract into or extend from the extension 21A. Preferably, the protrusion 34 is located at the left end 224 in a nonoperating position (see FIG. 3B). Further, the needle 31 is concealed by the cap 10 put on the extension 21A.

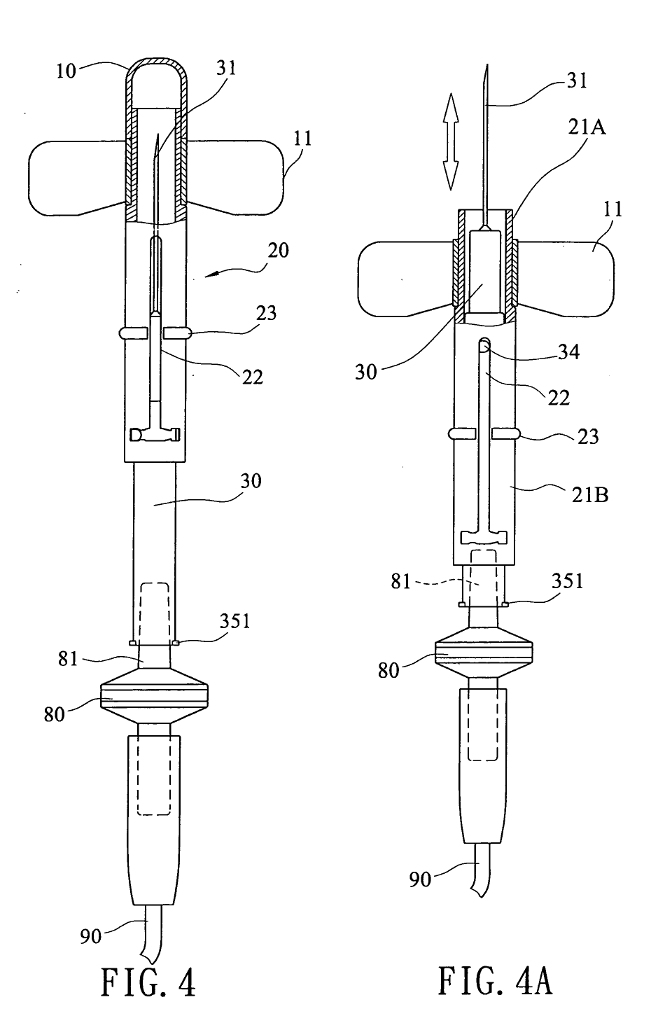

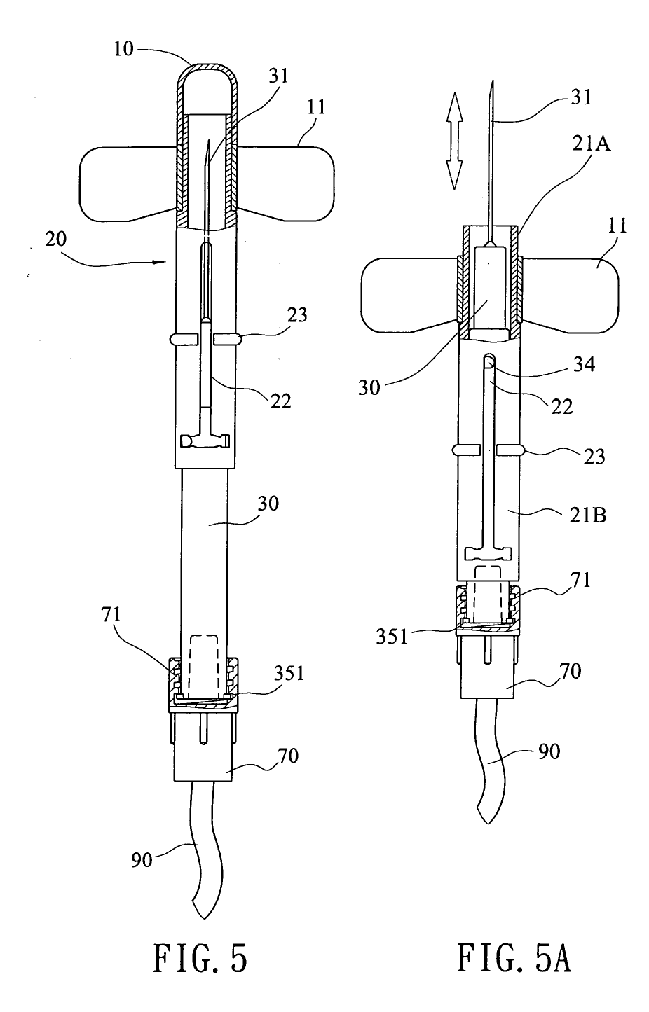

Referring to FIGS. 2 and 3A to 3C specifically, the cap 10 is removed prior to dispensing medicine. The protrusion 34 is disposed at the left end 224 in the nonoperating position (see FIG. 3B). Next, rightward slide the protrusion 34 to move to the turning point 22b (see FIG. 3A). Next, slide the protrusion 34 along the long bar of the slot 22 for projecting a pointed end 31a of the needle 31 from the extension 21A until the needle 31 is totally exposed when the protrusion 34 reaches the forward stop 22a, as shown in FIGS. 4A and 5A. The hand is completely behind the needle 31 during the above operation. Thus, a medical worker does not need to worry his/her hand being pricked accidentally by the needle 31.

After dispensing medicine, the medical worker can hold the slip-resistant ring 23 with one hand and hold the needle unit 30 (e.g., the latched members 351) with the other hand. Next, slide the protrusion 34 along the long bar of the slot 22 in a direction opposite to the above. Next, slide the protrusion 34 rightward to move into the cavity 225 after passing the turning point 22b and the shoulder 223 sequentially (see FIG. 3C). At this position, the protrusion 34 is locked since the straight section 341 has a length longer than that of the shoulder 223, i.e., a leftward movement of the protrusion 34 is prohibited. Also, the needle 31 is concealed in the barrel unit 20. Thus, the above safety characteristic can be maintained.

Referring to FIGS. 4 and 4A, in one configuration the sheath assembly can be mounted in an IV infusion set. In detail, a connector 81 of an adaptor 80 is inserted into the latched members 351 for coupling by snapping. Also, the adaptor 80 is coupled to an IV tubing 90. Thus, fluid can flow from the IV tubing 90 to the needle unit 30 via the adaptor 80 so as to dispense fluid into the body via the needle 31.

Referring to FIGS. 5 and 5A, in another configuration the sheath assembly can also be mounted in an IV infusion set. In detail, a connector 71 having internal threads of an adaptor 70 is threadedly secured to the latched members 351. Also, the adaptor 70 is coupled to an IV tubing 90. Thus, fluid can flow from the IV tubing 90 to the needle unit 30 via the adaptor 70 so as to dispense fluid into the body via the needle 31.

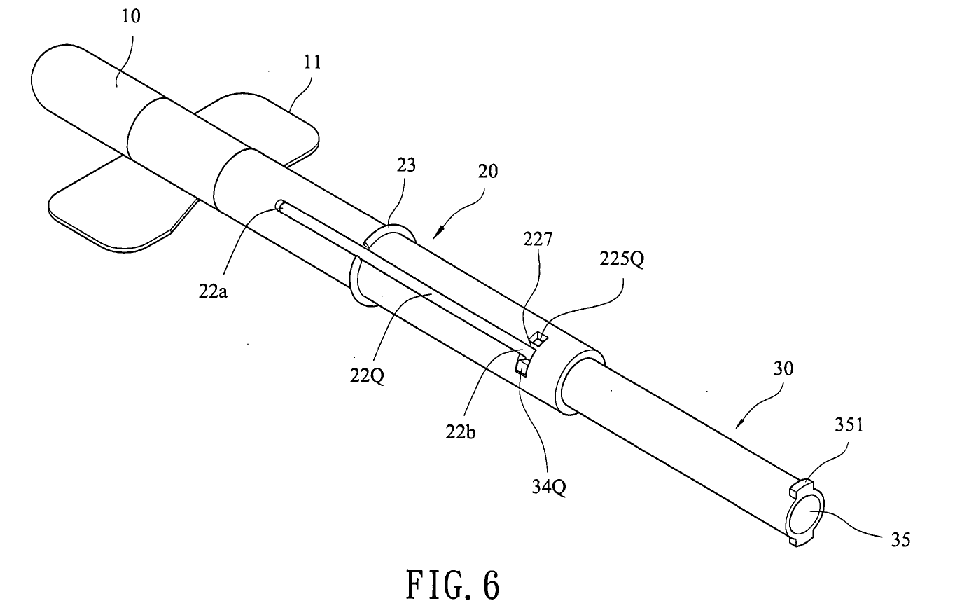

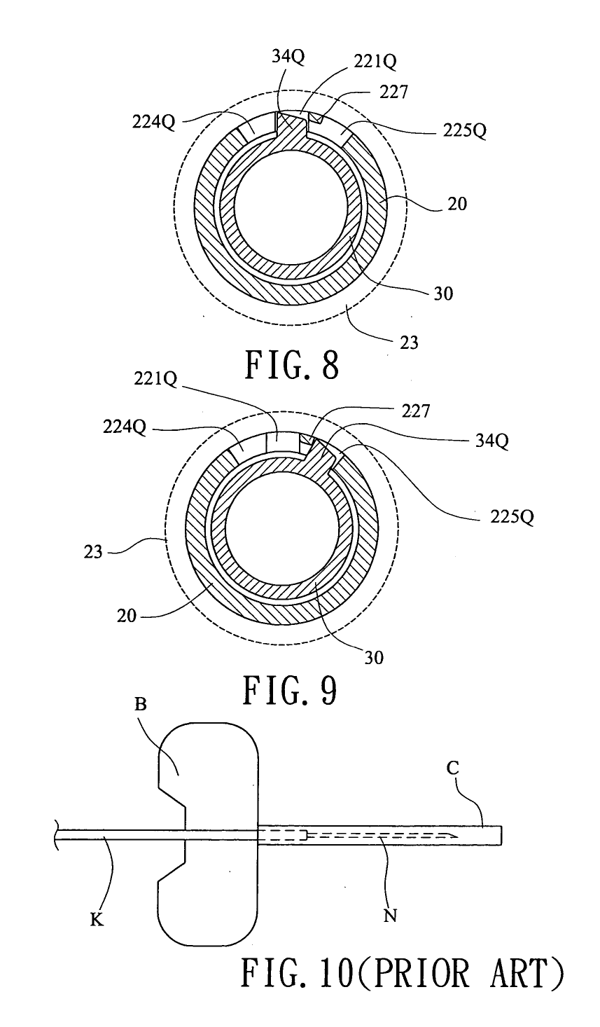

Referring to FIGS. 6 to 9, there is shown a second preferred embodiment of butterfly needle sheath assembly constructed in accordance with the invention. The second preferred embodiment substantially has same structure as the first preferred embodiment. The differences between the first and the second preferred embodiments, i.e., the characteristics of the second preferred embodiment are detailed below. The short bar 221Q of a T-shaped slot 22Q comprises a bridge 227 at a junction of the right portion thereof and the turning point 22b, a left end 224Q shaped to conform to a protrusion 34Q having a four-sided section and an arcuate top surface, and a right opening 225Q also having a shape conformed to that of the protrusion 34Q.

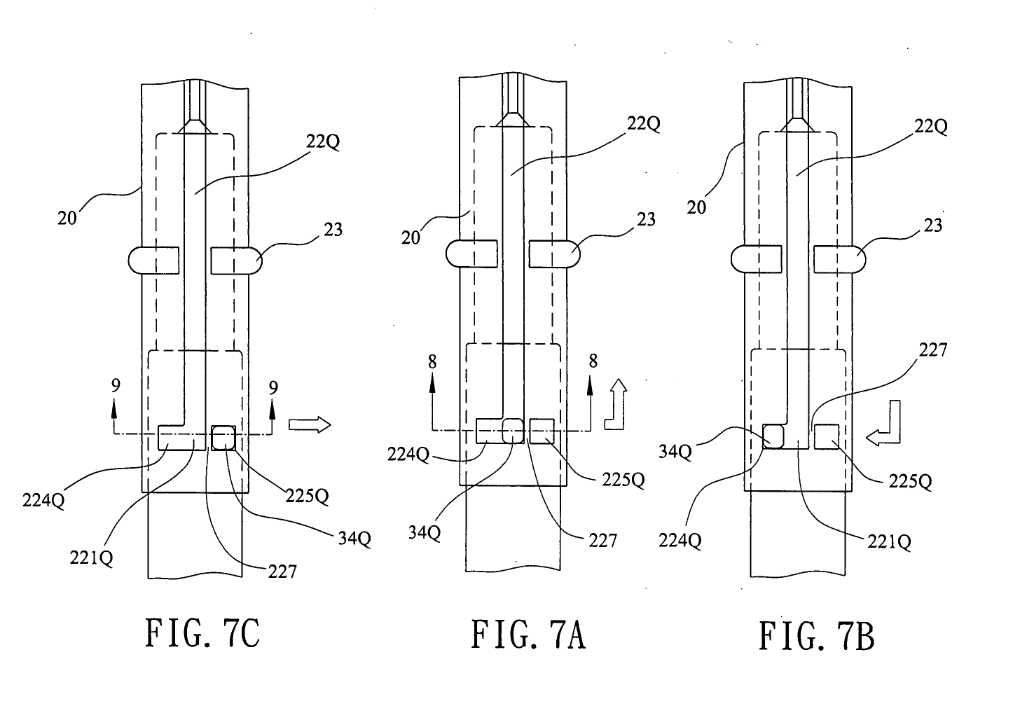

Referring to FIGS. 7A to 7C specifically, the cap 10 is removed prior to dispensing medicine. The protrusion 34Q is securely disposed at the left end 224Q in the nonoperating position. Next, rightward slide the protrusion 34Q to move to the turning point 22b. Next, slide the protrusion 34Q along the long bar of the slot 22Q for projecting the pointed end 31a of the needle 31 from the extension 21A until the needle 31 is totally exposed when the protrusion 34 reaches the forward stop 22a. After dispensing medicine, the medical worker can hold the slip-resistant ring 23 with one hand and hold the needle unit 30 with the other hand. Next, slide the protrusion 34Q along the long bar of the slot 22Q in a direction opposite to the above. Next, slide the protrusion 34Q rightward to move into the opening 225Q by passing the turning point 22b and the bridge 227 sequentially (see FIG. 7C). At this position, the protrusion 34Q is locked since the top of the protrusion 34Q is raised above the bridge 227, i.e., a leftward movement of the protrusion 34Q is prohibited.

In addition to the above advantages described in either embodiment, the invention also can completely prevent criminals from selling the butterfly needle sheath assemblies to hospitals again by simply cleaning or even disinfecting the used needles. In other embodiments, the protrusion can be provided in an inner surface of the barrel unit and the T-shaped slot can be provided on an outer surface of the needle unit as long as both material and structure of each of the barrel unit and the needle unit permit. Moreover, the pair of protrusion and T-shaped slot can provided in double for facilitating the sliding of the needle unit in the barrel unit.

While the invention herein disclosed has been described by means of specific embodiments, numerous modifications and variations could be made thereto by those skilled in the art without departing from the scope and spirit of the invention set forth in the claims.

What is claimed is:

1. A needle sheath assembly comprising:
a cap;
a wing unit including a hollow cylinder and two wings at both sides of the cylinder;
a barrel unit including a hollow barrel, a hollow, cylindrical extension at a forward end of the barrel, the extension being adapted to insert through the cylinder into the cap for coupling, and a T-shaped slot longitudinally formed on an outer surface of the barrel, a transverse bar of the slot including a right shoulder and a right cavity; and
a needle unit including a hollow plunger, a forward needle, two opposite latched members raised above an edge of a rear end, and a protrusion projected from an outer surface of the plunger wherein the protrusion is projected from the slot after inserting the needle unit in the barrel unit,
whereby removing the cap and sliding the protrusion from the transverse bar of the slot to a distal end of the slot will project the needle from the cylinder; and sliding the protrusion from the distal end of the slot to the right cavity through the right shoulder will lock the protrusion in the cavity.

2. The needle sheath assembly of claim 1, wherein the barrel unit further comprises a slip-resistant ring formed therearound.

3. The needle sheath assembly of claim 1, wherein the transverse bar further comprises a left throat adjacent a left end, the throat being adapted to fasten the protrusion at the left end in a nonoperating position.

4. The needle sheath assembly of claim 1, wherein the protrusion comprises a curved section and a straight section having a length longer than that of the shoulder for prohibiting a leftward movement of the protrusion.

5. The needle sheath assembly of claim 1, wherein the latched members are adapted to secure to IV infusion means.

6. The needle sheath assembly of claim 1, wherein the needle unit further comprises a forward extension with the needle protruded therefrom.

7. A needle sheath assembly comprising:
a cap;
a wing unit including a hollow cylinder and two wings at both sides of the cylinder;
a barrel unit including a hollow barrel, a hollow, cylindrical extension at a forward end of the barrel, the extension being adapted to insert through the cylinder into the cap for coupling, and a T-shaped slot longitudinally formed on an outer surface of the barrel, a transverse bar of the slot including a bridge at a junction of a right portion thereof and a right opening; and
a needle unit including a hollow plunger, a forward needle, two opposite latched members raised above an edge of a rear end, and a protrusion projected from an outer surface of the plunger wherein the protrusion is projected from the slot after inserting the needle unit in the barrel unit,
whereby removing the cap and sliding the protrusion from the transverse bar of the slot to a distal end of the slot will project the needle from the cylinder; and sliding the protrusion from the distal end of the slot to the right opening through the bridge will lock the protrusion in the right opening.

8. The needle sheath assembly of claim 7, wherein the barrel unit further comprises a slip-resistant ring formed therearound.

9. The needle sheath assembly of claim 7, wherein the protrusion comprises a top surface raised above each of the opening and the bridge when the protrusion is locked in the opening for prohibiting a leftward movement of the protrusion.

10. The needle sheath assembly of claim 7, wherein a left end of the transverse bar has a shape to fasten the protrusion therein in a nonoperating position.

11. The needle sheath assembly of claim 7, wherein the latched members are adapted to secure to IV infusion means.

12. The needle sheath assembly of claim 7, wherein the needle unit further comprises a forward extension with the needle protruded therefrom.

13. The needle sheath assembly of claim 7, wherein the protrusion has a four-sided section.
